(19) United States
(12) Patent Application Publication
(10) Pub. No.: US 2016/0206193 A1
(43) Pub. Date: Jul. 21, 2016

(54) FREQUENCY-DOMAIN INTERFEROMETRIC BASED IMAGING SYSTEMS

(71) Applicant: CARL ZEISS MEDITEC, Inc., Dublin, CA (US)

(72) Inventors: Tilman SCHMOLL, Dublin, CA (US); Matthew J. EVERETT, Livermore, CA (US)

(21) Appl. No.: 14/913,570

(22) PCT Filed: Aug. 21, 2014
(86) PCT No.: PCT/EP2014/002295

§ 371 Date: Feb. 22, 2016

Related U.S. Application Data
(60) Provisional application No. 61/869,256, filed on Aug. 23, 2013.
(60) Provisional application No. 62/010,367, filed on Jun. 10, 2014.
(60) Provisional application No. 62/031,619, filed on Jul. 31, 2014.

(51) Int. Cl. A61B 3/10; A61B 3/00; G01B 9/02

(52) U.S. Cl. CPC A61B 3/102; G01B 9/02044; G01B 9/02004; G01B 9/02091; A61B 3/0025

(57) ABSTRACT

Continuous time or non-integrating operation of an array of photosensitive elements for use as a detector in frequency domain interferometric imaging systems is described. Both swept-source and spectral domain embodiments are presented. Non-integrating or continuous mode camera operation enables much higher camera read-out rates compared to interferometric imaging systems using conventionally operated CMOS or CCD cameras.

### TECHNICAL FIELD

The present application relates to frequency domain interferometric systems, in particular a mode of operating the detector in such systems to enable higher speed operation.

### BACKGROUND

A wide variety of interferometric based imaging techniques have been developed to provide high resolution structural information in a wide range of applications. Optical Coherence Tomography (OCT) is a technique for performing high-resolution cross-sectional imaging that can provide images of samples including tissue structure on the micron scale in situ and in real time (see for example Huang et al. “Optical Coherence Tomography” Science 254 (5035): 1178 1991). OCT is an interferometric imaging method that determines the scattering profile of a sample along the OCT beam by detecting light reflected from a sample combined with a reference beam. Each scattering profile in the depth direction (z) is called an axial scan, or A-scan. Cross-sectional images (B-scans), and by extension 3D volumes, are built up from many A-scans, with the OCT beam moved to a set of transverse (x and y) locations on the sample.

Many variants of OCT have been developed where different combinations of light sources, scanning configurations, and detection schemes are employed. In time domain OCT (TD-OCT), the pathlength between light returning from the sample and reference light is translated longitudinally in time to recover the depth information in the sample. In frequency domain or Fourier domain OCT (FD-OCT), the broadband interference between reflected sample light and reference light is acquired in the spectral domain and a Fourier transform is used to recover the depth information. The sensitivity advantage of frequency-domain optical coherence tomography (OCT) over time-domain OCT is well established (see for example Choma et al. “Sensitivity advantage of swept source and Fourier domain optical coherence tomography,” Opt. Express 11, 2183-2189, 2003 and Leitgeb et al. “Performance of Fourier domain vs. time domain optical coherence tomography,” Opt. Express 11, 889-894, 2003).

There are two common approaches to FD-OCT. One is spectral domain OCT (SD-OCT) where the interfering light is spectrally dispersed prior to detection and the full depth information can be recovered from a single exposure. The second is swept-source OCT (SS-OCT) where the source is swept over a range of frequencies and detected as a function of time, therefore encoding the spectral information in time. In traditional point scanning or flying spot techniques, a single point of light is scanned across the sample. In parallel techniques, a series of spots, a line of light (line field), or a two-dimensional array of light (full-field or partial-field) are directed to the sample. A partial field system refers to a system that illuminates the sample with a field which is not large enough to illuminate the entire sample at once and detects the backscattered light with a 2D detector. In order to acquire an enface image or volume of the entire sample using a partial field illumination system, transverse scanning in at least one direction is required. A partial field illumination could be e.g. a low NA spot, a broad-line or an elliptical, square or rectangular illumination. In all cases, the resulting reflected light is combined with reference light and detected. Parallel techniques can be accomplished in TD-OCT, SD-OCT or SS-OCT configurations.

Several groups have reported on different parallel FD-OCT configurations (see for example Hiratsuka et al. “Simultaneous measurements of three-dimensional reflectivity distributions in scattering media based on optical frequency-domain reflectometry,” Opt. Lett. 23, 1420, 1998; Gajciar et al. “Parallel Fourier domain optical coherence tomography for in vivo measurement of the human eye,” Opt. Express 13, 1131, 2005; Povazay et al. “Full-field time-encoded frequency-domain optical coherence tomography” Optics Express 14, 7661-7669, 2006; Nakamura et al. “High-speed three-dimensional human retinal imaging by line-field spectral domain optical coherence tomography” Optics Express 15(12), 7103-7116, 2007; Lee et al. “Line-field optical coherence tomography using frequency-sweeping source” IEEE Journal of Selected Topics in Quantum Electronics 14(1), 50-55, 2008; Mujat et al. “Swept-source parallel OCT” Proceedings of SPIE 7168, 71681E, 2009; and Bonin et al. “In vivo Fourier-domain full-field OCT of the human retina with 1.5 million a-lines/s” Optics Letters 35, 3432-3434, 2010). In each case, a line or 2D camera comprising a plurality of photosensitive elements was used to acquire the OCT data. Typically these cameras use either complimentary metal-oxide-semiconductor (CMOS) or charge coupled device (CCD) photodetector arrays. CCD photodetector arrays inherently accumulate a charge on a capacitor, which is not read out until a control circuit triggers a charge transfer to a neighboring capacitor. This capacitor then dumps its charge into a charge amplifier, which converts the charge to a voltage which is digitized. In CMOS active pixels sensors (APS), photons hitting the photodiodes of the detector create a photocurrent, which is constantly transformed to a voltage. This voltage is then integrated by a capacitive transimpedance amplifier, over the exposure time before it is digitized. In such a configuration, CMOS detectors have to be reset at the end of each exposure time, before they can integrate again over the next exposure time. This reset takes some time, during which photons hitting the active detector area are not converted into an electrical signal. The time needed to reset the CMOS circuit is typically >1 μs. This sets a fundamental limit on the maximum line rates achievable with an integrating CMOS detector. At a line rate of 500 kHz and an ideal case of 1 μs dead time, already 50% of the line period is lost by the reset. Furthermore, the integration over a specific exposure time, acts as a low pass filter for the signal. This may be a disadvantage especially in the case of SS-OCT, since one is especially interested in the high frequency AC signal.

The related fields of Holoscopy, diffraction tomography, digital interference holography, Holographic OCT, and Interferometric Synthetic Aperture Microscopy (see for example Hillman et al. “Holoscopy—holographic optical coherence tomography: Optics Letters 36(13), 2390-2392, 2011; U.S. Pat. No 7,602,501; and Kim MK “Tomographic three-dimensional imaging of a biological specimen using wavelength-scanning digital interference holography” Optics Express 7(9) 305-310, 2000) are also interferometric imaging techniques that typically use photodetector arrays operated in an integrating mode for data collection.

A fast line scan camera in a SD-OCT system is disclosed by Potsaid et al. (Potsaid et al. “Ultrahigh speed Spectral/Fourier domain OCT ophthalmic imaging at 70,000 to 312,500 axial scans per second,” Optics Express 16, 15149-15169, 2008). Their system employed a Basler Sprint spL4096-140 km (Basler AG) line scan camera. They operated it at a maximum line rate of 312,500 lines per second. At this speed they were however only able to read out 576 pixels of the total array of 4096 pixels. The dead time of 1.2 μs corresponded at this speed to 37.5% of the total line period, which directly corresponds to a loss in sensitivity of 37.5%. Unless the light source is pulsed and its pulse length corresponds to the integration time and its average optical output power is kept constant compared to a corresponding continuous wave (CW) light source.

A fast point scanning SD-OCT system is disclosed by An et al. (“High speed spectral domain optical coherence tomography for retinal imaging at 500,000 A-lines per second,” Biomedical Optics Express 2, 2770-2783, 2011). In order to work around the camera dead time of the Basler Sprint spL4096-140 km line scan camera, they used two interleaved line scan cameras set to an individual line rate of 250,000 lines per second. By setting the exposure of each camera to 50% of the line period, they were able to reach a combined line rate of 500,000 lines per second. This way the effective exposure time was equal to the effective line period. Such a system suffers from several drawbacks. First of all the system cost is significantly increased by the need for duplicate cameras. Another significant drawback of this method is that in order to couple light from the sample to both cameras, one has to tolerate a loss in light efficiency on the path from the sample to the cameras, which directly results in a loss of sensitivity. Furthermore, in order to avoid image artifacts, one has to precisely match the alignment of the two spectrometers, which may be challenging for commercial systems. The authors mention that using the same system, they would be able to achieve a maximum combined line rate of 624,000 lines per second, when operating each camera at its maximum speed. Applying the same concept to a camera with a minimum dead time of 1 μs, a maximum combined line rate of 1 MHz with effectively 0% dead time could be envisioned. In principal, this method is also scalable to a higher number of cameras. For example, when setting the exposure time to a third of the line period and using three interleaved cameras, one would be able to triple the effectively dead time free line rate. The system complexity and costs however again significantly increase with each additional camera.

### SUMMARY

The present application proposes using a non-integrating camera design and mode of operation for frequency-domain interferometric optical imaging techniques. Non-integrating or continuous mode operation enables much higher camera read-out rates compared to interferometric imaging systems using conventionally operated CMOS or CCD cameras. Non-integrating camera operation should achieve camera line or frame rates in the MHz to GHz range, enabling A-scan rates of several GHz.

For frequency-domain interferometric based imaging techniques, scattered light returning from the sample is heterodyned with much more intense reference light which amplifies the signal. Therefore there is much more light on the detector, eliminating the need for integrating, and making high speeds possible. It is therefore sufficient to simply sample the photocurrent created by each photosensitive element in the detector or camera when light is incident on its photosensitive area. This operating mode, called continuous time mode or non-intergrating mode, allows for very high read-out rates, similar to the detection bandwidths of photodetectors employing single photodiodes or balanced photodetectors commonly used for point scanning SS-OCT. Envisioned line or frame read-out rates may theoretically reach several GHz, higher than the currently achieved 312,000 lines per second. So far line or frame read-out rates on this order have not been required by most imaging applications.

In particular, biomedical imaging methods usually expose only a limited amount of light onto the sample, which also limits the amount of light backscattered from the sample and therefore the maximum useful imaging speed. In many interferometric imaging modalities, this is however not an issue, due to the heterodyne amplification by the reference light, which is not exposed to the sample. To our knowledge detector arrays used for different kinds of frequency-domain interferometry based imaging have always been operated in an integrating mode. It has so far not been recognized that operating imaging detectors in a continuous time mode would be advantageous for point scanning SD-OCT, line field SD-OCT, multi-point scanning SS-OCT, line field SS-OCT, partial-field SS-OCT, or full-field SS-OCT. It is equally advantageous for related frequency domain interferometry based imaging techniques including but not limited to diffraction tomography, holographic OCT, interferometric synthetic aperture microscopy, and holoscopy.

Integrating cameras used for point scanning SD-OCT systems so far provided sufficiently high line rates. However, for parallel acquisition schemes, such as line field, partial-field, or full-field OCT or the corresponding parallel holographic OCT schemes, the camera read out rate of integrating cameras is a limiting factor for the maximum achievable imaging speed. For parallel OCT and parallel holographic OCT, especially high read out rates are required to minimize the impact of sample motion. Another distinct advantage of operating an array of photosensitive elements in a continuous time mode, is that it opens the possibility to process the generated electrical signal prior to its digitization, for example bandpass filtering of the signal to help suppress aliasing artifacts and increase the digitization dynamic range.

### BRIEF DESCRIPTION OF THE FIGURES

FIG. 1 shows a generalized holographic line field SS-OCT system.

FIG. 2 shows a schematic of an integrating mode pixel configuration. For simplicity the figure only shows a single pixel of a larger array of pixels.

FIG. 3 shows a schematic of a continuous mode pixel configuration. For simplicity the figure only shows a single pixel of a larger array of pixels.

FIG. 4 shows a generalized point-scanning SD-OCT system.

FIG. 5 illustrates one embodiment of a partial-field SS-OCT holosocopic system.

FIG. 6 illustrates the prior art of how a camera of an OCT system is commonly connected to a processor

FIG. 7 illustrates an interferometric imaging system arrangement where the camera is attached to the processor.

FIG. 8 illustrates an interferometric imaging system arrangement where the camera is attached to an FPGA, which is again attached to the processor.

FIG. 9 illustrates an imaging system in which a memory cache is included directly on each pixel or with the camera so that the data does not need to be transferred to the computer in real time

### DETAILED DESCRIPTION

A frequency-domain interferometric imaging system embodying a camera in continuous time mode will now be described. The detailed description is primarily focused on holographic SS-OCT systems but as will be discussed, the invention described herein could be applied to any type of camera based frequency-domain interferometric imaging system.

In this specification, we use the term photosensitive element to refer to an element that converts electromagnetic radiation (i.e. photons) into an electrical signal. It could be a photodiode, phototransistor, photoresistor, avalanche photodiode, nano-injection detector, or any other element that can translate electromagnetic radiation into an electrical signal. The photosensitive element could contain, on the same substrate or in close proximity, additional circuitry, including but not limited to transistors, resistors, capacitors, amplifiers, analog to digital converters, etc. When part of a detector, the photosensitive element is also commonly referred to as pixel, sensel or photosite. A detector or camera can have an array of photosensitive elements or pixels.

A typical holographic line-field SS-OCT system is illustrated in FIG. 1. Light from a tunable light source 100 is collimated by a spherical lens 101a. A cylindrical lens 102a creates a line of light from the source, and the light is split into sample arm and reference arm by a beam splitter 103. A scanner 200 can adjust the transverse location of the line of light on the sample 104. A pair of spherical lenses 101b and 101c images the line onto the sample 104. The light in the reference arm is transferred back to a collimated beam by a cylindrical lens 102c before it is focused on a mirror 105 by a spherical lens 101d and reflected by said mirror 105. By the time it passes the beam splitter 103 the second time, the reference light travelled close to the same optical path length as the sample arm light did. At the beam splitter 103, light reflected back from the reference arm and backscattered in the sample arm are recombined and coherently interfere with each other. The light, which has been modulated by this interference is then directed towards line detector 106 comprising an array of photosensitive elements after passing through a lens 101e. In a holographic line field SS-OCT system as illustrated here, the line of light on the line detector 106 is significantly defocused along the line. The additional astigmatism is introduced by a cylindrical lens 102b in the detection path as described in U.S. Patent Publication No. 2014/0028974 Tumlinson et al. “Line-field Holoscopy” hereby incorporated by reference. The electrical signals from the line detector 106 are transferred to the processor 109 via a cable 107. The processor 109 may contain a field-programmable gate array (FPGA) 108, which performs some, or the entire OCT signal processing steps, prior to passing the data on to the host processor 109. The processor is operably attached to a display 110 for displaying images of the data. The sample and reference arms in the interferometer could consist of bulk-optics, photonic integrated circuits (PIC), planar waveguides, fiber-optics or hybrid bulk-optic systems and could have different architectures such as Michelson, Mach-Zehnder or common-path based designs as would be known by those skilled in the art. Light beam as used herein should be interpreted as any carefully directed light path.

Line field SS-OCT systems typically acquire several A-scans in parallel, by illuminating the sample with a line and detecting the backscattered light with a line scan camera. While the tunable laser sweeps through its optical frequencies, several hundred line acquisitions are required in order to be able to reconstruct a cross-section with a reasonable depth (>500 microns) and resolution. Sample motion occurring within one sweep can significantly alter the image quality. It is therefore desirable to keep the sweep time as short as possible. The minimum sweep time is, in contrast to point scanning SS-OCT systems, currently not limited by the tunable laser. Instead it is currently limited by the maximum line rate of available line scan cameras. Faster line scan cameras may therefore directly impact the success of high speed line field SS-OCT.

A significant limitation for the maximum speed of line scan cameras is the reset time required by CMOS detectors. CMOS APS sensors are typically operated in a so called integration mode. They accumulate during each exposure time a charge, e.g. on a capacitor. At the end of each exposure time, and before a new charge can be accumulated, the capacitor has to be reset. This reset lasts typically in the order of >=1 μs. This significantly limits the maximum achievable line rate. At a line rate of 500 kHz and an ideal case of 1 μs dead time, already 50% of the line period is lost by the reset. This is especially critical because during this reset time, none of the photons hitting the active area of the photodiode are converted to an electric signal. This directly results in a loss in signal. The reset time is therefore also called the “dead time” of the detector.

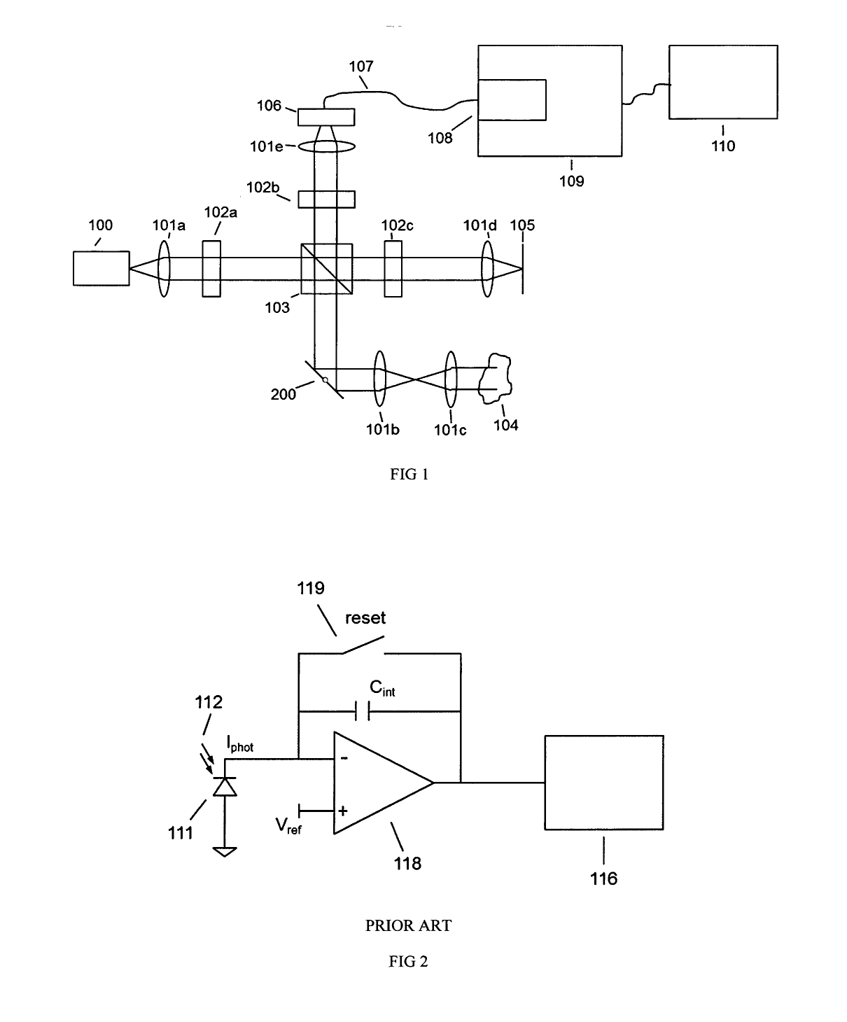

FIG. 2 shows a schematic of a single pixel configured in integration mode. For simplicity the figure only shows a single pixel of a larger array of pixels. The incident light 112 hitting the photodiode 111 generates a photocurrent, which is then integrated over the exposure time by a capacitive transimpedance amplifier 118. At the end of each exposure time its output voltage is digitized by an analog to digital converter (ADC) 116. Before a new charge can be integrated, the pixel is set in reset mode by closing a switch 119.

Here we propose using a different type of camera configuration for camera based frequency-domain interferometry imaging systems, and in a preferred embodiment for holographic line-field SS-OCT systems. Instead of operating an array of photosensitive elements in an integrating mode as described above, the array is operated in a continuous time mode. In this mode the charge is not integrated over an exposure time. Instead, the photogenerated charge of each individual photosensitive element is converted into a steady-state photocurrent, which is sampled as a function of time. Such an operation mode is known in other imaging techniques (see for example Ricquier et al., “Active Pixel CMOS Image Sensor with On-Chip Non-Uniformity Correction, ” IEEE Workshop on CCDs and Advanced Image Sensors, Dana Point, Calif., Apr. 20-22 1995; Fossum ER, “CMOS Image Sensors: Electronic Camera On A Chip,” Electron Devices, IEEE Transactions on 44, 1689-1698, 1997; Huang et al., “Current-Mode CMOS Image Sensor Using Lateral Bipolar Phototransistors,” IEEE Transactions on Electron Devices 50, 2003; Bourquin et al. “Video-rate optical low-coherence reflectometry based on a linear smart detector array” Optics Letters 25, 102-104, 2000; Bourquin et al. “Optical coherence topography based on a two-dimensional smart detector array” Optics Letters 26, 512-514, 2001; Laubscher et al. “Video-rate three-dimensional optical coherence tomography” Optics Express 10, 429-435, 2002; Serov et al. “Laser Doppler perfusion imaging with a complementary metal oxide semiconductor image sensor” Optics Letters 27(5), 300-302, 2002; and Samuel Osei Achamfuo-Yeboah “Design and Implementation of a CMOS Modulated Light Camera” University of Nottingham PhD Thesis 2012).

For most imaging applications it has not been desirable to operate an image sensor in a continuous time mode, because without integration, higher light intensities are necessary in order to achieve good quality images. Especially in biomedical imaging applications the sample may not be exposed to very high light intensities. While non-integrating cameras have been used in time domain interferometric systems, to our knowledge, it has not been recognized that it would be advantageous to operate cameras in frequency-domain interferometric imaging systems in such a mode. Interferometric imaging systems profit from the heterodyne amplification by the reference light. All camera based frequency-domain interferometic imaging systems, including but not limited to point scanning SD-OCT, multi-point scanning SD-OCT, line field SD-OCT, line field SS-OCT, partial-field SS-OCT, or full-field SS-OCT could profit from using cameras which are configured in a continuous time mode. We however want to emphasize that especially parallel techniques, where the speed of available cameras is currently limiting imaging speed and therefore image quality, will profit from employing cameras configured in a continuous time mode.

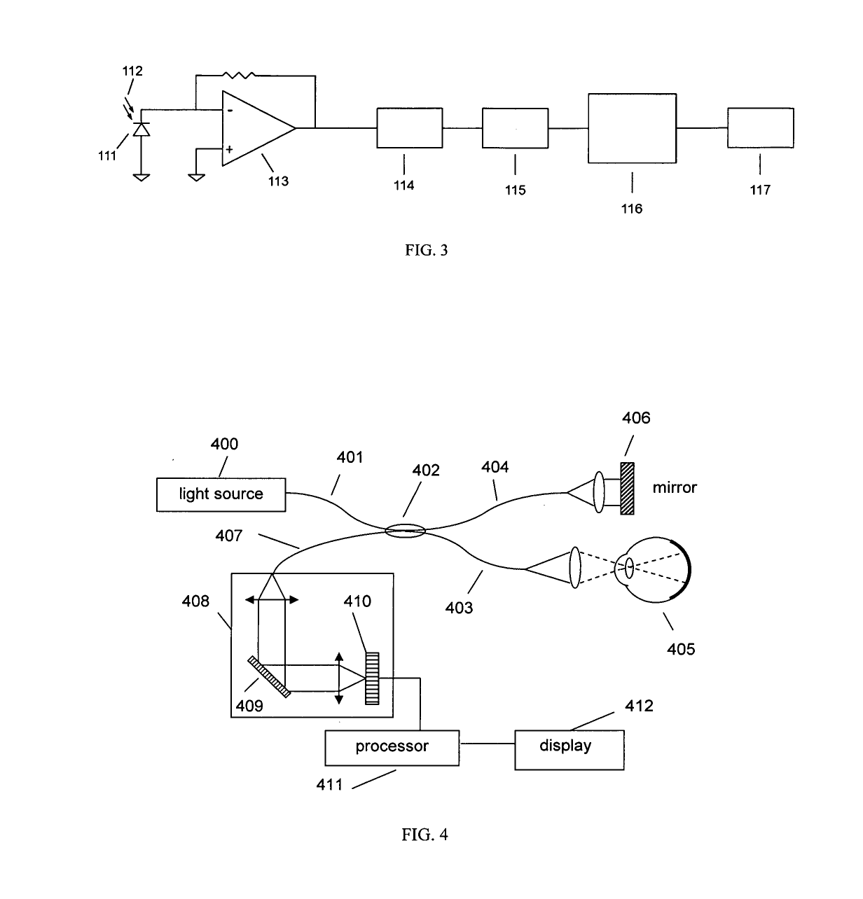

FIG. 3 shows a schematic of a single pixel configured in a non-integrating mode. For simplicity the figure only shows a single pixel of a larger array of pixels. The incident light 112 hitting the photodiode 111 generates a photocurrent. This photocurrent is constantly amplified and converted to a voltage by a transimpedance amplifier 113. The voltage signal can then be high-pass filtered 114 and low-pass filtered 115 before it gets digitized by an ADC 116. The digital data can then be temporarily stored in a first in first out (FIFO) buffer 117 before it is transferred to for example an external processor or a FPGA for further data processing.

To collect a volume of data with a line field system as is illustrated in FIG. 1 containing a camera operated in a continuous time mode, one would arrange multiple pixels, schematically illustrated in FIG. 3, to create a linear array 106. Using this linear array one would sample the light incident on the array typically at least several hundred times while the source 100 is swept over a range of frequencies. In a preferred embodiment, the source is swept linearly in wavenumber, k. The system could also be operated with a k-clock or the data could be digitally resampled to create data that is linear in k. In between sweeps, the scanner 200 directs the sample light to a slightly different transverse location on the sample 104, before the line array 106 again samples the light incident on the linear array. This procedure is repeated until a volume of the desired size is scanned.

In one embodiment, the reverse biased photodiodes in a detector array are connected to individual operational amplifiers, which convert the photocurrents into voltages and amplify them. The voltage signal can then be further processed, e.g. by high- and low-pass filters. This will allow suppressing aliasing artifacts, caused by the finite digitization frequency. It could also allow suppressing the DC term, so one may make better use of the full dynamic range of the digitization. After these signal conditioning steps, the voltages of each photodiode can then be digitized by individual analog to digital converters. Such a configuration would allow for a very high degree of parallelization. Alternatively, the voltages can also be time multiplexed and supplied to one or several common high speed ADCs. Such a configuration avoids the need for a large number of individual ADCs, but, may on the other hand, not be able to achieve similar line rates. In order to avoid a high number of individual operational amplifiers, one may also choose to time multiplex the photocurrents and supply them to a common operational amplifier and a common ADC.

The described continuous time mode photodiode array configuration has the advantage that no reset is needed between detections, and very high detection bandwidths in the MHz to GHz range become feasible. The described circuitry may be realized by integrated circuits on the same chip as the photodiodes or on a separate module.

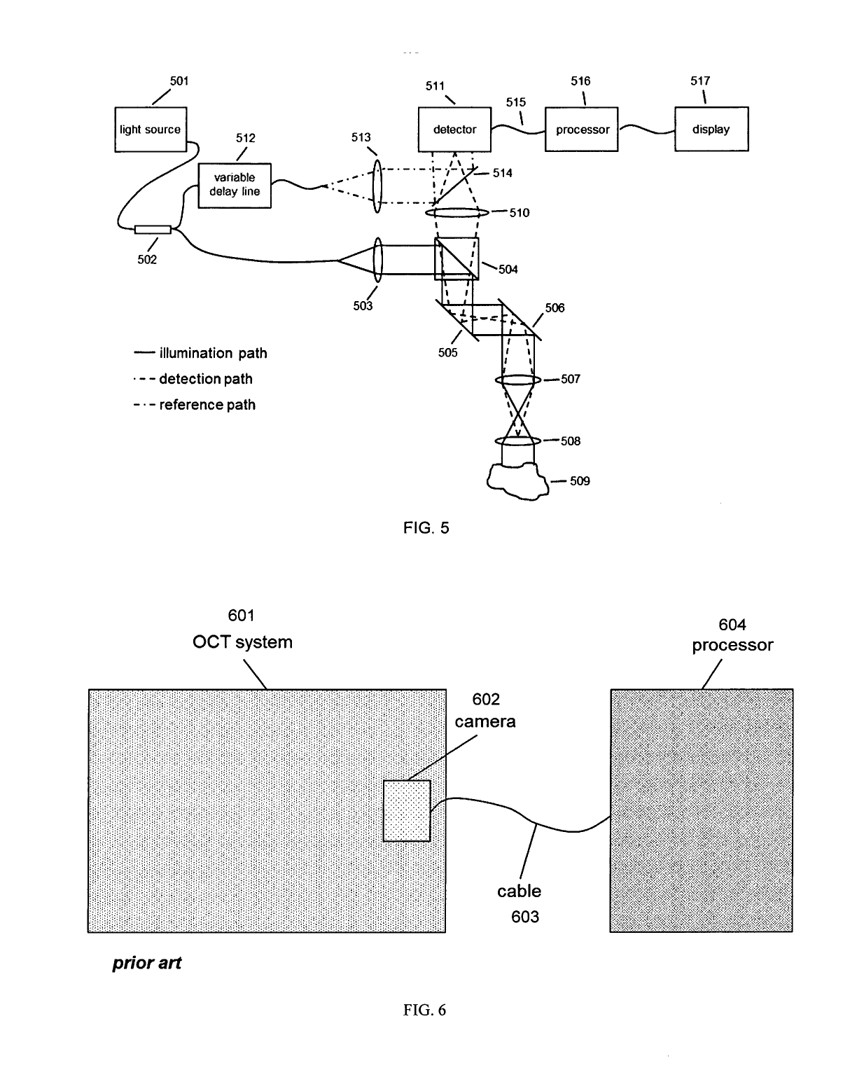

Such a camera design should be advantageous for holographic line field SS-OCT as described in detail above with respect to FIG. 1. In a similar fashion it should also be advantageous for partial field SS-OCT holoscopy systems. One embodiment of a swept source based partial-field holoscopy system is illustrated in FIG. 5. Light from a tunable light source 501 is split into sample light and reference light by a fused coupler 502. The sample light is collimated by a spherical lens 503 and reflected by a beam splitter 504. Two scanners 505 and 506 can adjust the transverse location of the line of light on the sample 509. A pair of spherical lenses 507 and 508 creates an area illumination on the sample 509. In the detection path (path from sample to the detector), the light backscattered by the sample is detected in a conjugate plane of the pupil of lens 508. Lens 507 images the pupil plane to the scanners 506 and 507 and lens 510 relays this image onto the detector 511. The reference light first passes a variable delay line 512 which allows to adjust the optical path length difference between the sample and reference light. The reference light is then collimated by a spherical lens 513 and reflected onto the detector 511 by a beam splitter 514. The beam splitter 514 is oriented in a way to create an angle between reference and sample light.

Typically the variable delay line 512 is adjusted so that sample and reference light travel close to the same optical distance before they coincide on the detector 511, where they coherently interfere. In addition to the interference modulation as a function of optical wavenumber, spatial interference fringes across the detector can be introduced by the angle between reference arm and sample arm.

The electrical signals from the detector 511 are transferred to the processor 516 via a cable 515. The processor 516 may contain a field-programmable gate array (FPGA), a digital signal processor (DSP), or an application specific integrated circuit (ASIC), which performs some, or the entire holoscopy signal processing steps, prior to passing the data on to the host processor 516. The processor is operably attached to a display 517 for displaying images of the data. The sample and reference arms in the interferometer could consist of bulk-optics, photonic integrated circuits, fiber-optics or hybrid bulk-optic systems and could have different architectures such as Michelson, Mach-Zehnder or common-path based designs as would be known by those skilled in the art.

Partial-field SS-OCT systems typically acquire several A-scans in parallel, by illuminating the sample with a two-dimensional area and detecting the backscattered light with a 2D detector array of photosensitive elements. While the tunable laser sweeps through its optical frequencies, several hundred detector acquisitions are required in order to be able to reconstruct a volume with a reasonable depth (>500 μm) and resolution. In order to acquire a volume, the illumination area on the sample is scanned across the sample using two 1-axis scanners (505 and 506) and multiple spatially separated volumes are acquired. Alternatively a single 2-axis scanner could be used to fulfill the task of the two 1-axis scanners.

Continuous mode or non-integrating mode camera operation could also be used in standard point scanning SD-OCT. FIG. 4 shows a basic block diagram for a point scanning spectrometer based SD-OCT system. The light source 400, typically a superluminescent diode (SLD), provides broad bandwidth light to a short length of an optical fiber 401 to an input port of a fiber optic coupler 402, which splits the incoming light beam into the two arms of an interferometer. The two arms each have a section of optical fiber 403 and 404 that guides the split light beam from the fiber coupler 402 to the eye of a patient 405 and a reference reflector 406 respectively. For both the sample arm and the reference arm, at the terminating portion of each fiber, there may be a module containing optical elements to collimate or focus or scan the beam. The returned light waves from the sample 405 and the reference reflector 406 are directed back through the same optical path of the sample and reference arms and are combined in fiber coupler 402. A portion of the combined light beam is directed through a section of optical fiber 407 from the fiber coupler 402 to a spectrometer 408. Inside the spectrometer, the light beam is dispersed by a grating 409 and focused onto a detector array 410. The collected data is sent to a processor 411 and the resulting processed data can be displayed on a display 412 or stored in memory for future reference and processing. Although the system of FIG. 1 includes a reflective reference arm, those skilled in the art will understand that a transmissive reference arm could be used in its place. As in the linefield holoscopy example of FIG. 1, the sample and reference arms in the interferometer could consist of bulk-optics, fiber-optics or hybrid bulk-optic systems and could have different architectures such as Michelson, Mach-Zehnder or common-path based designs as would be known by those skilled in the art. Light beam as used herein should be interpreted as any carefully directed light path.

A 2D continuous time photodiode array may also be used in a similar way for a line field SD-OCT system, a partial-field SS-OCT system or a full-field SS-OCT system and provide the same advantages. The complexity of such detectors however scales with the number of photodiodes. A 2D photodiode array with a high number of photodiodes therefore exhibits considerably higher complexity as compared to a linear photodiode array.

The use of such high speed cameras generates very large amounts of data. In traditional camera-processor configurations, the camera represents a stand-alone device, which handles the light collection, the conversion to an electric signal and some signal conditioning, before it transfers the signals to a processor (e.g. personal computer (PC)) over a wired connection. FIG. 6 illustrates such a configuration, where an OCT system 601 contains a camera 602, which is connected via a cable 603 to an external processor 604. Typically used connections include but are not limited to USB, CameraLink, CoaXpress, or Ethernet connections, but wireless connections could also be used. The transfer step represents another bottleneck in the imaging process, which may limit the speed of a high speed line-field, partial-field, or full-field interferometric imaging system. In a preferred embodiment of the present invention, the camera may be attached directly to the PC, e.g. via Peripheral Component Interconnect Express (PCIe) interface.

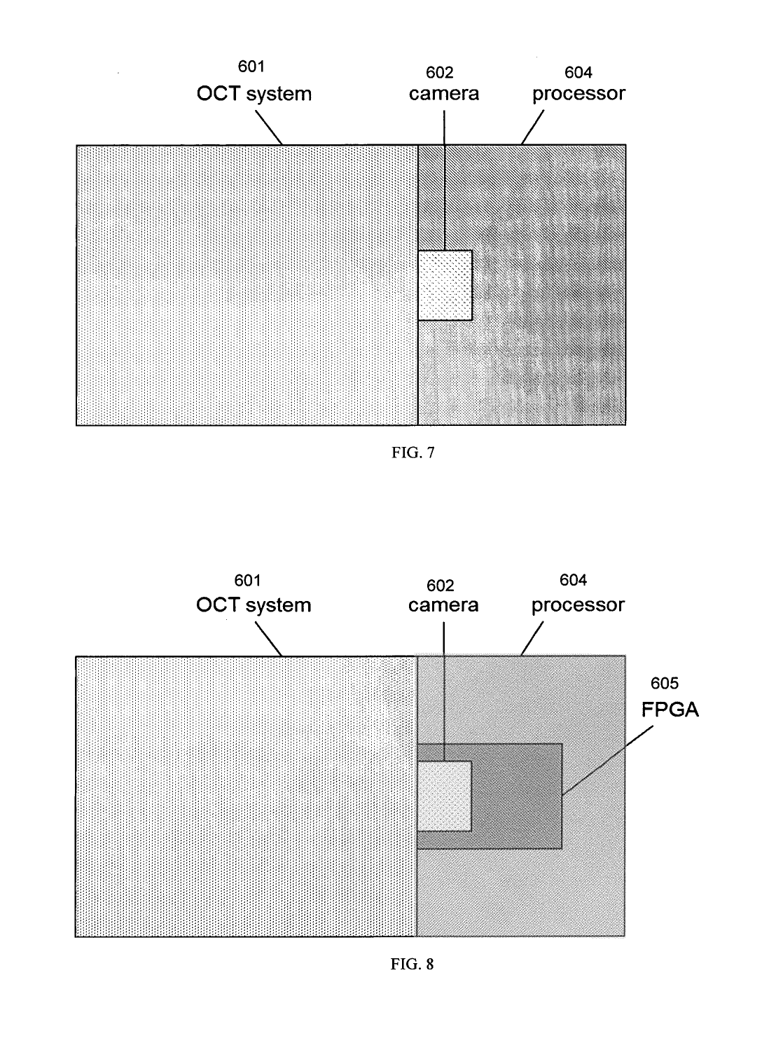

FIG. 7 shows a configuration, where an OCT system 601 is placed in close proximity to the processor 604, which holds the camera 602. In an alternative embodiment, the camera may be directly attached to a field-programmable gate array (FPGA), e.g. via a FMC connector, which handles some or all of the OCT processing steps. After these processing steps the data would be transferred from the FPGA to the host computer, e.g. via PCIe.

FIG. 8 illustrates a configuration, where an OCT system 601 is placed in close proximity to the processor 604, which holds the camera 602, which is directly attached to an FPGA 605, used for signal processing. By positioning the camera and processor in close proximity, one avoids the need of a data transfer via a cable or wireless connection with limited bandwidth. The use of faster transfer methods such as high speed multi-lane PCIe, becomes feasible.

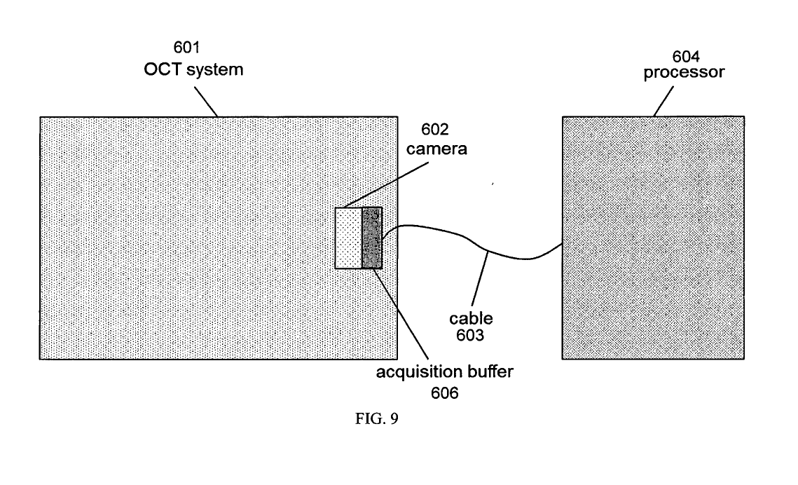

FIG. 9 illustrates another possible embodiment based on the prior art system of FIG. 6 with OCT system 601 having camera 602 connected via cable 603 to processor 604, but wherein a memory cache or acquisition buffer 606 is included directly on each pixel(FIG. 3) or with the camera 606 so that the data does not need to be transferred to the computer in real time. Total acquisition time, especially in ophthalmology is typically limited to a few seconds. This is because motion artifacts increase with increasing imaging time and patient comfort significantly decreases with increasing imaging time. Having a memory buffer within the camera, which can hold the data of a several second long acquisition, could therefore help to circumvent the bottleneck of data transfer between the camera and processor. One would be able to quickly store the acquired data in the buffer during the acquisition and then accept a slower data transfer to the processor.

Although various applications and embodiments that incorporate the teachings of the present invention have been shown and described in detail herein, those skilled in the art can readily devise other varied embodiments that still incorporate these teachings. The following references are hereby incorporated by reference:

### PATENT REFERENCES

EP Patent No. 0519105 Hubert et al. “Photodiode Array”

U.S. Patent Publication No. 2012/0261583 Watson et al. “High-sensitivity, high-speed continuous imaging system”

U.S. Patent Publication No. 2014/0028974 Tumlinson et al. “Line-field Holoscopy”

U.S. Pat. No. 7,602,501 Ralston et al. “Interferometric synthetic aperture microscopy”

WO 2012/143113 Hillman et al. “Method for Optical Tomography”

U.S. Pat. No. 7.643,155 Marks et al. “Partially coherent illumination for inverse scattering full-field interferometric synthetic aperture microscopy”

U.S. Pat. No. 8,480,579 Serov et al. “Instrument and method for high-speed perfusion imaging”

U.S. Pat. No. 6,263,227 Boggett et al. “Apparatus for imaging microvascular blood flow”

PCT Publication No. WO 03/063677 Serov et al. “Laser Doppler perfusion imaging with a plurality of beams”

GB Patent No. 2413022 Serov et al. “Laser Doppler perfusion imaging using a two-dimensional random access high pixel readout rate image sensor”

PCT Publication No. WO 2013/160861 Andre et al. “Optical Coherent Imaging Medical Device”

### NON-PATENT LITERATURE

An et al. “High speed spectral domain optical coherence tomography for retinal imaging at 500,000 A-lines per second,” Biomedical Optics Express 2, 2770-2783, 2011

Blazkiewicz et al, “Signal-to-noise ratio study of full-field Fourier-domain optical coherence tomography” Applied Optics 44(36):7722 (2005).

Bonin et al. “In vivo Fourier-domain full-field OCT of the human retina with 1.5 million a-lines/s” Optics Letters 35, 3432-3434, 2010.

Bourquin et al. “Video-rate optical low-coherence reflectometry based on a linear smart detector array” Optics Letters 25, 102-104, 2000.

Bourquin et al. “Optical coherence topography based on a two-dimensional smart detector array” Optics Letters 26, 512-514, 2001.

Choma et al. “Sensitivity advantage of swept source and Fourier domain optical coherence tomography,” Opt. Express 11, 2183-2189, 2003.

Choi et al. “Fourier domain optical coherence tomography using optical demultiplexers imaging at 60,000,000 lines/s” Optics Letters 33, 1318-1320, 2008.

Egan et al. “Full-field optical coherence tomography with a complimentary metal-oxide semiconductor digital signal processor camera” Optical Engineering 45(1), 015601, 2006.

Fossum, “CMOS Image Sensors: Electronic Camera On A Chip,” Electron Devices, IEEE Transactions on 44, 1689-1698, 1997.

Franke et al. “High Resolution Holoscopy” Proceedings of APIE Volume 8213, 821324 2012.

Gajciar et al. “Parallel Fourier domain optical coherence tomography for in vivo measurement of the human eye,” Opt. Express 13, 1131, 2005.

Haeusler et al., “Coherence Radar” and “Spectral Radar-new tools for dermatological diagnosis,” Journal of Biomedical Optics 3, 21-31, 1998.

Hillman et al. “Common approach for compensation of axial motion artifacts in swept-source OCT and dispersion in Fourier-domain OCT” Optics Express 20(6), 6761-6776, 2012.

Hillman et al. “Holoscopy—holographic optical coherence tomography: Optics Letters 36(13), 2390-2392, 2011.

Hiratsuka et al. “Simultaneous measurements of three-dimensional reflectivity distributions in scattering media based on optical frequency-domain reflectometry,” Opt. Lett. 23, 1420, 1998.

Huang et al. “Optical Coherence Tomography” Science 254 (5035): 1178 1991.

Huang et al., “Current-Mode CMOS Image Sensor Using Lateral Bipolar Phototransistors,” IEEE Transactions on Electron Devices 50, 2003.

Kim M K “Tomographic three-dimensional imaging of a biological specimen using wavelength-scanning digital interference holography” Optics Express 7(9) 305-310, 2000.

Kim M K “Wavelength-scanning digital interference holography for optical section imaging” Optics Letters 24(23), 1693-1695, 1999.

Laubscher et al. “Video-rate three-dimensional optical coherence tomography” Optics Express 10, 429-435, 2002.

Lee et al. “Line-field optical coherence tomography using frequency-sweeping source” IEEE Journal of Selected Topics in Quantum Electronics 14(1), 50-55, 2008.

Leitgeb et al. “Performance of Fourier domain vs. time domain optical coherence tomography,” Opt. Express 11, 889-894, 2003

Marks et al. “Inverse Scattering for frequency-scanned full-field optical coherence tomography” Journal of the Optical Society of America A 24(4), 1034-1041, 2007.

Mujat et al. “swept-source parallel OCT” Proceedings of SPIE 7168, 71681E, 2009.

Nakamura et al. “High-speed three-dimensional human retinal imaging by line-field spectral domain optical coherence tomography” Optics Express 15(12), 7103-7116, 2007.

Potcoava et al. “Optical Tomography for biomedical applications by digital interference holography” Meas. Sci. Technol. 19, 074010, 2006.

Povazay et al. “Full-field time-encoded frequency-domain optical coherence tomography” Optics Express 14, 7661-7669, 2006.

Potsaid et al. “Ultrahigh speed Spectral/Fourier domain OCT ophthalmic imaging at 70,000 to 312,500 axial scans per second,” Optics Express 16, 15149-15169, 2008.

Ricquier et al., “Active Pixel CMOS Image Sensor with On-Chip Non-Uniformity Correction, ” IEEE Workshop on CCDs and Advanced Image Sensors, Dana Point, Calif., Apr. 20-22 1995.

Samuel Osei Achamfuo-Yeboah “Design and Implementation of a CMOS Modulated Light Camera” University of Nottingham PhD Thesis 2012.

Serov et al., “High-speed laser Doppler perfusion imaging using an integrating CMOS image sensor,” Opt. Express 13, 6416-6428, 2005.

Serov et al. “Laser Doppler perfusion imaging with a complementary metal oxide semiconductor image sensor” Optics Letters 27, 300-302, 2002.

Serov et al. “High-speed laser Doppler perfusion imaging using an integrating CMOS image sensor” Optics Express 13, 6416-6428, 2005.

Yu et al., “Variable tomographic scanning with wavelength scanning digital interference holography,” Opt. Comm. 260, 462-468 (2006).

Zvyagin et al “Full-field Fourier domain optical coherence tomography” Proc. SPIE 5690 2005.

1-14. (canceled)

15. A frequency-domain parallel interferometry based imaging system for imaging a light scattering object, said system comprising:
a frequency-swept light source for generating a beam of radiation;
a beam divider for separating the beam into reference and sample arms, wherein the sample arm contains the light scattering object to be imaged;
optics to apply said beam of radiation to the light scattering object to be imaged;
a detector including an array of photosensitive elements wherein the photosensitive elements are operated in continuous-time mode where the charge of each photosensitive element is converted to a continuous photocurrent that is sampled in time;
return optics for combining light scattered from the object and light from the reference arm and directing the combined light towards the array of photosensitive elements;
a processor for generating an image in response to the sampled photocurrent.

16. An interferometry based imaging system as recited in claim 15, wherein the photocurrent is continuously amplified and converted to a voltage prior to being sampled.

17. An interferometry based imaging system as recited in claim 15, wherein the interferometry based imaging technique is optical coherence tomography.

18. An interferometry based imaging system as recited in claim 15, wherein the interferometry based imaging system is one of holoscopy, diffraction tomography, digital interference holography, Holographic OCT, and Interferometric Synthetic Aperture Microscopy.

19. An interferometry based imaging system as recited in claim 15, wherein the beam of radiation is focused to a line on the object and wherein the array of photosensitive elements is linear.

20. An interferometry based imaging system as recited in claim 15, wherein the beam of radiation is focused into a two-dimensional area on the object and wherein the array of photosensitive elements is two-dimensional.

21. An interferometry based imaging system as recited in claim 20, wherein the system is a full-field system.

22. An interferometry based imaging system as recited in claim 20, wherein the system is a partial-field system.

23. An interferometry based imaging system as recited in claim 15, wherein at least part of the processor is located external to the source, beam divider, optics, detector and return optics and wherein the detector passes data directly to the external processor.

24. An interferometry based imaging system as recited in claim 15, further comprising a field-programmable gate array (FPGA) for performing some of the image generating functions.

25. An interferometry based imaging system as recited in claim 15, wherein the detector operates at a line rate greater than 320,000 lines per second.

26. An interferometry based imaging system as recited in claim 15, wherein the return optics include a cylindrical lens.

27. An interferometry based imaging system as recited in claim 15, wherein the photosensitive elements are photodiodes.

28. A frequency-domain parallel interferometry based imaging system for imaging a light scattering object, said system comprising:
a light source for generating a beam of radiation;
a beam divider for separating the beam into reference and sample arms, wherein the sample arm contains the light scattering object to be imaged;
optics to apply said beam of radiation to the light scattering object to be imaged;
a detector including an array of photosensitive elements wherein the photosensitive elements are operated in continuous-time mode where the charge of each photosensitive element is converted to a continuous photocurrent that is sampled in time, said detector further including optics for spectrally dispersing light across the array;
return optics for combining light scattered from the object and light from the reference arm and directing the combined light towards the array of photosensitive elements;
a processor for generating an image in response to the sampled photocurrent

29. An interferometry based imaging system as recited in claim 28, wherein the photocurrent is continuously amplified and converted to a voltage prior to being sampled.

30. An interferometry based imaging system as recited in claim 28, wherein the interferometry based imaging technique is optical coherence tomography.

31. An interferometry based imaging system as recited in claim 28, wherein the interferometry based imaging system is one of holoscopy, diffraction tomography, digital interference holography, Holographic OCT, and Interferometric Synthetic Aperture Microscopy.

32. An interferometry based imaging system as recited in claim 28, wherein the beam of radiation is focused to a line on the object and wherein the array of photosensitive elements is two-dimensional.

33. An interferometry based imaging system as recited in claim 28, wherein the beam of radiation is focused into a two-dimensional area on the object and wherein the array of photosensitive elements is two-dimensional.

34. An interferometry based imaging system as recited in claim 33, wherein the system is a full-field system.

35. An interferometry based imaging system as recited in claim 33, wherein the system is a partial-field system.

36. An interferometry based imaging system as recited in claim 28, wherein at least part of the processor is located external to the source, beam divider, optics, detector and return optics and wherein the detector passes data directly to the external processor.

37. An interferometry based imaging system as recited in claim 28, further comprising a field-programmable gate array (FPGA) for performing some of the image generating functions.

38. An interferometry based imaging system as recited in claim 28, wherein the detector operates at a line rate greater than 320,000 lines per second.

39. An interferometry based imaging system as recited in claim 28, wherein the return optics include a cylindrical lens.

40. An interferometry based imaging system as recited in claim 28, wherein the photosensitive elements are photodiodes.
